(19) United States
(12) Patent Application Publication
(10) Pub. No.: US 2002/0086846 A1
(43) Pub. Date: Jul. 4, 2002

(54) Gene transfer to renal glomerular cells

(76) Inventors: Xuehai Ye, Arlington, VA (US); Patricio E. Ray, Potomac, MD (US)

Correspondence Address: Law Offices of Dr. Melvin Blecher, 4329 Van Ness St., NW, Washington, DC, 20016-5625, US

(21) Appl. No.: 09/972,956

(22) Filed: Oct. 10, 2001

Related U.S. Application Data
(60) Provisional application No. 60/246,041, filed on Nov. 6, 2000.

(51) Int. Cl. A61K 48/00; C12N 15/861

(52) U.S. Cl. 514/44; 424/93.2; 435/456; 435/320.1

(57) ABSTRACT

The present invention relates to a method of introducing exogenous genes into renal glomerular cells using an adenoviral vector. The method allows for efficient infection of renal glomerular cells with the viral vector, and provides for high-level expression of the exogenous gene in the infected cells. The invention can be used for both in vitro and in vivo applications in humans and laboratory animal models.

### FIELD OF INVENTION

The current invention concerns methods of gene transfer into renal tissue. More specifically, the invention is a method of introducing genetic material into renal glomerular cells at high efficiency using an adenoviral vector.

### BACKGROUND OF INVENTION

The rapid development of gene transfer technology provides an opportunity to develop treatments for various inherited or acquired renal disorders. Several strategies have been developed to deliver foreign genes into different segments of the nephron using viral or non-viral vectors, as well as genetically modified renal cells.

Foreign gene expression in renal tubular epithelial cells and interstitial cells has been attempted using intra-arterial, intra-ureteral or intra-parenchchymal injections of cationic liposomes (Tomita et al. Biochem Biophys Res Commun 186: 129 (1992); Isaka et al.. J Clin Invest. 92:2597 (1993); Imai et al. Exp. Nephrol 5:112 (1997); Lai et al. Gene Ther 4: 426 (1997); recombinant retroviruses (Bosch et al. Exp Nephrol 1: 49 (1993); adenovirus (Moullier et al. Kidney Int 45:1220 (1994); Heikkila et al. Gene Ther 3: 21 (1996); Zhu et al. Gene Ther 3:298 (1996); Zhu et al. Kidney Int 52:992 (1997), or adeno-associated virus (Lipkowitz et al. J Am Soc Nephrol 10:1908 (1999). However, the targeting of renal glomerular cells with viral vectors has been more problematic. So far, several genes have been expressed in renal glomeruli of rodents either by infusing genetically modified renal cells into renal circulation (Kitamura et al. J Clin Invest 94:497 (1994); Kitamura Proc Nat Acad Sci 93:7387 (1996); Kitamura, J Clin Invest 100:1394 (997), or by using liposomes conjugated with viral proteins (Tomita et al. 1992 above; Akami et al. Transplantation Proc 26:1315 (1994). However, the liposome-mediated transgene expression was transient, while genetically modified renal cells trapped in the glomeruli could potentially induce additional biological effects and/or glomerular injury.

Adenoviral vectors offer several advantages over other methods for the development of renal gene transfer techniques in small animal models (Lipkowitz et al. Am J Kidney Dis 28:475 (1996); Kelly et al. Am J Physiol 276:F-1 (1999). The viral vectors can be easily produced at high titers, have a wide host range, and infect non dividing cells (Lipkowitz et al, 1996, above; Kelly et al, 1999, above). However, previous studies have shown that a single injection of adenovirus into the renal artery of rodents did not result in any gene transfer to glomeruli (Moullier, et al. 1994, above; Zhu et al. 1996, above). These studies questioned the ability of renal glomerular cells to be infected by adenoviral vectors and raised the question of whether these cells have the necessary receptors to be infected with these viral vectors.

Recently, we demonstrated that the liver plays a key role in determining the clearance of adenoviruses from the systemic circulation (Ye et al., Hum Gene Therap, in press (2000)). We found that by bypassing the liver circulation, the retention time and concentration of circulating viruses were increased, and that subsequently glomerular endothelial cells could be effectively infected with adenovirus. These findings led us to the concept that a prolonged exposure to the virus, following isolation of the circulation from that of the body, might be required for an effective infection of renal glomerular cells. We have now reduced this concept to practice by determining whether rat renal glomerular cells could be efficiently infected by a recombinant adenoviral vectors carrying the galactosidase gene (AdCBlacZ). We also determined the use of this technique under conditions which avoided renal injury. In addition, we explored the potential application of this technique to the treatment of human glomerular diseases, we tested the in vitro sensitivity of different human glomerular cell types to the infection of adenoviral vectors. Our results are detailed below.

### SUMMARY OF THE INVENTION

The present invention provides for the transfer of a selected gene or genes into renal glomerular cells.

In one aspect of the invention, an effective amount of adenovirus vector carrying the genetic material to be transferref is slowly infused into the renal artery for an effective period of time under conditions which protect the kidney from ischemic injury.

In one embodiment, the virus is introduced into the renal artery through the superior mesenteric artery (SMA) with a concurrent clamping on the aorta above and below the SMA to restrict blood flow from SMA into the aorta, and the kidney is protected from ischemic damage by maintaining the kidney at a reduced temperature during viral infusion.

In another embodiment, the adenovirus vector carries a control element that preferentially expresses the transgene in renal glomerular cells.

In still another embodiment, a plastic tube is inserted into the femoral vein, advanced through the vena cava into the renal vein, and secured in place by a suture tied around the renal vein. This tubing allows the viruses that are not taken up by the kidney to be released outside of the blood circulation, thereby preventing the infection of extra-renal tissues by the viral vector.

The present invention provides the first demonstration of efficient gene transfer in rat renal glomerular cells without inducing significant glomerular injury. It is a simple method that can be used to create small animal models to study the effect of foreign gene transfer into renal glomeruli. It may also be used to test novel therapies for different human renal diseases.

### BRIEF DESCRIPTION OF THE DRAWINGS

FIG. 1 is a sketch depicting the possible routes for infusing a single kidney.

FIG. 2. A photograph depicting adenovirus-mediated lacZ expression in rat kidney.

FIG. 3. A microphotograph depicting adenovirus-mediated lacZ expression in glomeruli.

FIG. 4. A microphotograph depicting adenovirus-mediated lacZ gene expression in a primary culture of human glomerular cells.

### DETAILED DESCRIPTION OF THE INVENTION

We have discovered a method of gene transfer into renal glomerular cells of animals, and an embodiment of this method that minimizes escape of the adenoviral vector for the gene into tissues other than renal glomeruli.

The E1-deleted recombinant adenovirus carrying the Escherichia coli lacZ gene encoding β-galactosidase (designated Ad.CBlacZ) was generated as described before (Kozarsky et al. Som Cell Mol Genet 19:449 (1993). In this viral vector, the lacZ gene expression is controlled by the cytomegalovirus (CMV) enhancer and the chicken β-actin gene promoter. The particle/plaque forming unit (pfu) ratio of the virus stock used in the experiments was 100. The method of the invention is not limited to the lacZ gene; other suitable genes include: green fluorescence protein; erythropoietin; CD2-associated protein; nephrin; growth factors such as basic fibroblast growth factor, vascular endothelial growth factor, transforming growth beta, platelet-derived growth factor, granulocyte-macrophage colony-stimulating factor; and, chemikines such as monocyte-chemoattractant protein-1, macrophage-inflammatory protein 1 and 2, cytokine-induced neutrophil chemoattractants, and RANTES.

To produce a rodent animal model, male Sprague-Dawley rats (100-150 g) may be purchased from Taconic Farms (Germantown, N.Y.). All animals should be housed in a temperature controlled room with a 12 h on/12 h off lighting schedule for at least 48 hours before use. Standard rat chow and water are given ad libitum

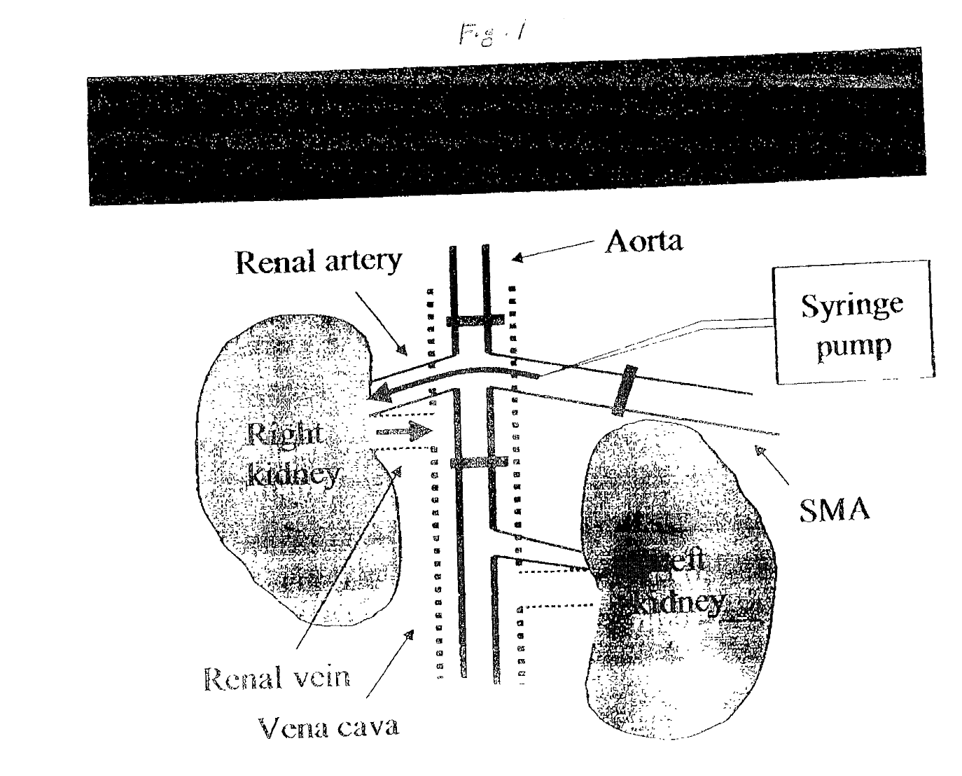

To administer the adenoviral vector, animals, e.g., rats, may be injected intramuscularly with 20,000 units of penicillin, anesthetized with ketamine (70 mg/kg, ip) and xylazine (7 mg/kg, i.p.), and undergo surgical exposure of the right kidney, the aorta and the right renal blood vessels. The right renal blood flow may be interrupted by clamping the aorta above and below the right renal artery and the superior mesenteric artery (SMA) (FIG. 1). This setting selectively excludes the right kidney without interrupting the blood circulation through the left kidney and allows infusion of virus into the right kidney through the SMA. A 27-gauge winged infusion needle is inserted into the SMA and fixed in place with a microaneurism clamp. 1.5 ml of Ad.CBlacZ (1×10¹¹ or 5×10 10¹¹ particle/ml in phosphate buffered saline (PBS) containing 5 units of heparin/ml are slowly infused into the right kidney with a Razel A-99 syringe pump at a flow rate of 0.1 ml/min. The flow rate may be adjusted according to the size of the animal without undo experimentation. By “slow flow rate” is meant within the context of this invention a rate such that the infusion is complete within about 15 to 120 minutes.The right kidney is packed with ice during the infusion to minimize ischemic damage. Renal circulation is reestablished at the end of infusion. Experimental animals, e.g., rats, are sacrificed at appropriate intervals, e.g., day 3 and 21, post-viral infusion (i.p.). Gene transfer efficiency is determined by lacZ staining of tissue sections, and the left kidney may be used as an internal negative control. LacZ expression in the liver may also examined for possible spreading of virus. To determine β-galactosidase (lacZ) activity in tissue samples and cell cultures, renal cells and frozen renal sections (10 μm) may be fixed in 0.5% glutaraldehyde at room temperature for 10 min, washed with PBS, and stained for 2 h at 37° C. in PBS containing 5mM K₃Fe(CN)₆, 5mM K₄Fe(CN)₆, 1mM MgCl₂, and 1 mg/ml 5-bromo-4-chloro-3-indolyl-β-D-galactopyranoside (X-gal). The tissue sections are counter-stained with hematoxylin and mounted for microscopic evaluation. The intensity of lacZ staining is evaluated by counting lacZ positive cells in ten microscopic fields using a 10× lens.

The intrarenal arterioles from control kidneys and kidneys may be infused with adenoviral vectors and carefully dissected under direct stereoscopic visualization. The vascular trees may be fixed in 0.5% glutaraldehyde at room temperature for 20 min, washed with PBS, and stained for 1 h for lacZ activity as described above.

For immunochemistry, kidney tissues may be fixed in 10% neutral buffered formalin, embedded in paraffin, and sectioned at 4 μm. The tissue sections are then heated twice for 5 min each in 0.01 M sodium citrate (pH 6.0) in a microwave oven (2450 mHz, 850 W) to augment antigen retrieval. Endogenous peroxidase activity may be blocked by treating the sections with 3% H₂O₂ in 100% methanol for 10 min. Immunostaining may be performed with a commercial streptavidin-biotin-peroxidase complex HistostainTM SP kit (Zymed, Minn.) according to the manufacture's instructions. The peroxidase substrate chromagen may be aminoethylcarbazole. To determine the degree of renal histologic injury, renal sections are counterstained with either hematoxylin and eosin (H&E), period acid/Schiff (PAS) or the Masson stain following standard techniques (Masson, J Tech Med 12:75 (1929). The Masson staining may be carried out using the Diagnostics Trichrome at Stain Kit from Sigma as described by the manufacturer's instructions. The following antibodies may be used for immunohistochemistry studies: (1) a rabbit anti-human factor VIII related antigen (vWF) (Dako, Copenhagen, Denmark), enzymatic predigestion with trypsin was used for greater staining intensity and uniformity; (2) a monoclonal anti-smooth-muscle-α-actin (Sigma, St. Louis, Mo.); (3) a mouse anti-rat monocytes/macrophages (ED1) monoclonal antibody (Chemicon International Inc., Temecula, Calif., USA); (4) a biotinylated proliferating cell nuclear antigen (PCNA) monoclonal antibody (clone PC10) (Zymed, Minn.). Negative controls may be performed either by omitting the primary antibody or using a preimmune rabbit serum or a mouse IgG for the polyclonal and monoclonal antibody, respectively. Immunohistochemical staining was quantitated by counting the number of positive glomerular and tubular cells showing positive staining with each specific antibody in 10 microscopic field in each renal section.

Glomerular injury may be evaluated by counting the percentage of glomeruli with sclerotic and inflammatory lesions using the H&E, PAS and Masson staining, as well as by immunohistochemical staining with specific antibodies against von Willbrand's factor (vWF), α-actin, PCNA, ED-1. For each section, a suitable number, e.g., 50, glomeruli are examined. PCNA or ED1 positive cells on kidney sections are counted under a microscope using a 100× field. Kidney sections from three animals are examined at each time point and positive cells in 10 randomly chosen microscopic fields are counted for each kidney section. Tubulo-interstitial injury is graded (0−5+) based on the presence of tubular cellularity, basement membrane thickening, dilatation, atrophy, presence of tubular cast, sloughing or interstitial widening as follows: grade 0: no changes present; grade 1:<10%; grade 2: 10-25%; grade 3: 25-50%; grade 4: 50-75%; and grade 5: 75-100% tubulo-interstitial changes. For each renal section, the entire cortical and medullary region may be evaluated in 10 fields of 1 mm² and a mean score per renal section was calculated.

Primary human glomerular endothelial cells may be obtained from the Applied Cell Biology Research Institute, Seattle, Wash. (Dr. Carl Soderland). These cells are positive for expression of von Willenbrand Factor (vWF) and are grown in the endothelial cell culture medium from Cell Systems (Seattle, Wash.) containing recombinant human epidermal growth factor (rhEGF, 10 ng/ml), bovine brain extract, heparin (10 μg/ml), amphotericine B (50 ng/ml), gentamycin (50 μg/ml), and 5% fetal bovine serum (FBS). Human glomerular podoctyes are isolated from renal glomeruli derived from a kidney considered unsuitable for transplantation. Briefly, the renal cortex is minced into 1-mm fragments and passed through graded series of metal sieves. The resulting material containing approximately 95% glomeruli by microscopic examination is incubated with collagenase (type IV, Sigma) at 37° C. for 15 minutes. Glomeruli are then pelleted, resuspended in culture media and seeded on different culture dishes as previously described (Izevbigie et al. Ped. Nephrol, in press 2000). Colonies of glomerular epithelial cells (GEC) are selected using cloning rings, while all other cells attached to the culture dishes may be discarded using a cell scraper. GEC are grown in culture media KGM2-BulleKit from BioWhittaker Inc. (Walkersville Md.). Expanded colonies of GEC which show positive immunostaining with a monoclonal antibody against the podocyte specific antigen GLEEP-1 (available from Dr. Roger Wiggins, University of Michigan, Ann Arbor Mich.), and positive staining with an antibody against WT-1 (Dako, Copenhagen, Denmark), wmay be used for the infection studies. Human mesangial cells are isolated from renal glomeruli as we have previously described (Ray et al. 1999, above; Izevbigie et al. 2000, above). Mesangial cells show a polygonal shape, stain positive for F actin using Bodipy-phallacidin (Molecular Probes, Eugene Oreg.), lack the close cell-cell contact typical of endothelial cells or epithelial cells, and form ridge composed of multiple layers of cells surrounding regions of monolayers. Human mesangial cells (HMC) do not stain positive with anti-cytokeratin or anti-human vWF antibodies (Dako, Copenhagen, Denmark) but did stain positive with an anti-myosin antibody (Zymed, Minn.). HMC express a high number of AngII type 1 receptors (Bₘₐₓ of approximately 70 fmol/10⁵ cells) and contracted in response to AngII (100 nM) when seeded onto the surface of a silicone rubber substratum.

Renal urinary tubular epithelial cells (RUTEc) are derived from a child with HIVAN. These cells are expanded, and characterized as we have previously described (Ray et al. Kidney Int 53:1217 (1998); Izevbigie et al. 2000, above). These cells stain positive with a cytokeratin antibody, and showed negative staining with antibodies against the following: WT-1, GLEEP, and vWF. RUTEc cell are cultured in REGM BulleKit culture media from Clonetics. All cells were used between passages 2-4. To determine the susceptibility of each cell type to adenoviral infections, the endothelial cell culture medium may be used for all cell types. This culture medium supports the growth of all human cell types used in this study.

To study adenovirus infection of cultured cells, cells are plated in 6-well culture dishes at a density of 5×10⁵ cells/well. Recombinant adenoviruses are added to the culture medium 24 h later at a multiplicity of infection (MOI) of 0.4, 4, and 40 pfu/cell. The cells are fixed 24 h after the infection and stained for lacZ activity as described above.

Results are expressed as the mean value _+SD. Differences between groups are compared by Students't test when the distribution of the data followed a Gaussian distribution or by the non-parametric Kruskal-Wallis test when the distribution of the data did not follows a Gaussian distribution. When more that two means are compared, the difference may be measured by one way analysis of variance followed by multiple comparison using the Students-Neuman-Keul's test. P-values less than 0.05 are considered statistically significant.

The practice of the present invention will employ, unless other wise indicated, conventional methods of histology, virology, microbiology, immunology, and molecular biology within the skill of the art. Such techniques are explained fully in the literature. All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

In describing the present invention, the following terms will be employed, and are intended to be defined as indicated below:

1. A “gene” or “coding sequence” or a sequence which “encodes” a particular protein, is a nucleic acid molecule which is transcribed (in the case of DNA) and translated (in the case of mRNA) into a polypeptide in vitro or in vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even,synthetic DNA sequences. A transcription termination sequence will usually be located 3′ to the gene sequence

2. The term “transgene” refers to the gene of interest that is introduced into host cells by the adenoviral vector.

3. The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present so long as the selected coding sequence is capable of being replicated, transcribed and translated in an appropriate host cell. Such control elements are well known to those of average skills in molecular biology.

4. The phrase “deliver a gene” or “transfer a gene” or “gene transfer” refers to methods or systems for reliably introducing genetic material encoding the gene of interest into host cells, such as into renal glomerular cells. Such methods can result in transient or long term expression of the gene of interest from the transferred genetic material.

5. The term “adenoviral vector” or “recombinant adenovirus' refers to the modified adenovirus that is used as a vector to deliver a gene. The genome of an adenovirus can be manipulated such that it encodes and expresses a gene product of interest but is inactivated in terms of its ability to replicate in a normal lytic viral life cycle. Suitable adenoviral vectors derived from the adenovirus sera type 5 and other sera types (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome. Moreover, the carrying capacity of the adenoviral genome for foreign DNA is large relative to other gene delivery vectors. Most replication-defective adenoviral vectors currently in use are deleted for all the viral genes but retain only the elements required in cis for replication and packaging, allowing for about 35kb of “genomic space” to incorporate transgenes and regulation systems.

6. The element “slow infusion” is intended to mean an infusion of the vector that takes about 15 mins. to less than 120 mins. to complete.

7. The element “single-pass infusion” is intended to mean an infusion of the vector as a single bolus, as opposed to the use of closed circuit infusion of the vector.

The following examples are meant merely to exemplify several embodiments, and should not be interpreted as limiting the scope of the claims, which are delimited only by the specification.

### EXAMPLE 1

### Prolonged Renal Infusion

This example describes the procedure for slowly infusing a recombinant adenovirus into the renal circulation (FIG. 1). Male Sprague-Dawley rats (100-150 g) were injected intramuscularly with 20,000 units of penicillin, anesthetized with ketamine (70 mg/kg, ip) and xylazine (7 mg/kg, ip) and underwent surgical exposure of the right kidney, the aorta and the right renal blood vessels. The right renal blood flow was interrupted by clamping the aorta above and below the right renal artery and the superior mesenteric artery (SMA)(FIG. 1). This setting selectively excluded the right kidney without interrupting the blood circulation through the left kidney and allowed infusion of virus into the right kidney through the SMA. A 27-gauge winged infusion needle was inserted into the SMA and fixed in place with a microaneurism clamp. 1.5 ml of recombinant adenovirus in phosphate buffered saline (PBS) containing 5 units of heparin/ml were slowly infused into the right kidney with a Razel A-99 syringe pump at a flow rate of 0.1 ml/min. The right kidney was packed with ice during the infusion to minimize ischemic damage. Renal circulation was reestablished at the end of infusion. The abdominal cavity was closed with sutures. The animal was placed on a warm pad to recover from the anesthesia and was returned to its cage after recovery.

### EXAMPLE 2

### Adenovirus-mediated LacZ Expression In Glomerular Cells

This example describes the efficiency, persistence and distribution of adenovirus-mediated lacZ expression in the infused kidney.

The virus-infused animals were sacrificed at day 3 and 21 post-viral infusion (p.i.). Gene transfer efficiency was determined by lacZ staining of tissue sections, and the left kidney was used as an internal negative control. LacZ expression in the liver, spleen, heart, lung and intestine was also examined for possible spreading of virus.

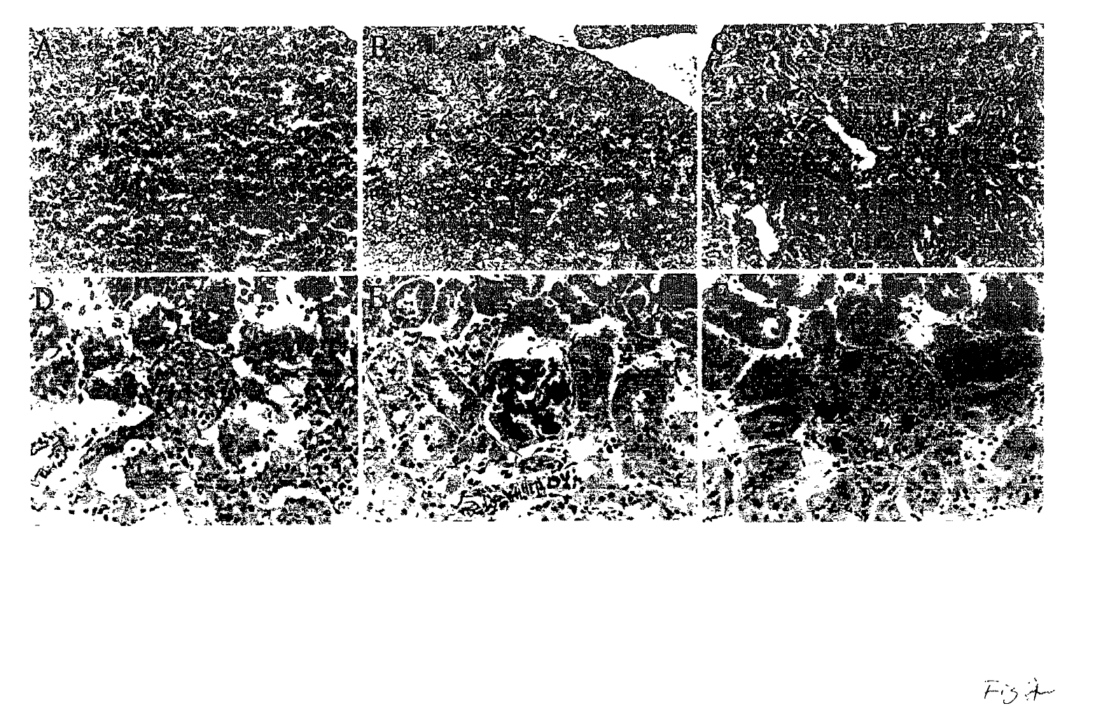

Ad.CBlacZ (1.5 ml, 5×10¹¹ particle/ml) was slowly infused into the right kidney of 100-150 g male Sprague-Dawley rats at a flow rate of 0.1 ml/min . The animals were sacrificed at day 3 or 21 after viral infusion. The efficiency of gene transfer was E determined by lacZ staining of tissue sections. FIG. 2 panels A and D show the control left kidney at day 3 post-viral infusion (p.i.); panels B and E show infused right kidney at day 3 p.i.; panels C and F show infused right kidney at day 21. Representative pictures are shown. Original magnification: 40× for panel A, B and C; 200× for panel D, E and F.

Adenovirus-mediated transgene expression at day 3 post viral infusion is summarized in Table 2 and FIG. 2. Expression was detected in the liver of all animals treated with the virus, effective gene transfer to kidney was achieved only in animals receiving a 15-min infusion with the higher viral dose. LacZ expression in the infused kidney (right kidney) was limited to the glomeruli (FIG. 2B) and occasionally in the endothelial cells of blood vessels (data not shown). The percentage of glomeruli expressing the lacZ gene varied from 30-70% among treated animals. However, the diffuse staining pattern of lacZ made it difficult to determine the exact cell types that were positive for lacZ expression. A close examination at higher magnification of renal sections which showed minimal staining demonstrated that the glomerular endothelial cells were predominately positive (FIG. 2E). Under the current experimental condition, there was no lacZ expression in control contralateral left kidneys in all the treated animals (FIGS. 2A, 2D) suggesting that β-galactosidase was produced in the kidney, as opposed to being produced in the liver and trapped in the kidney (Zhu et al. 1997, above). The adenovirus-mediated lacZ expression in the glomeruli was still detectable at day 21 post viral infusion, although the intensity of lacZ staining and the number of lacZ-positive glomeruli were significantly decreased compared to day 3 samples (FIG. 2).

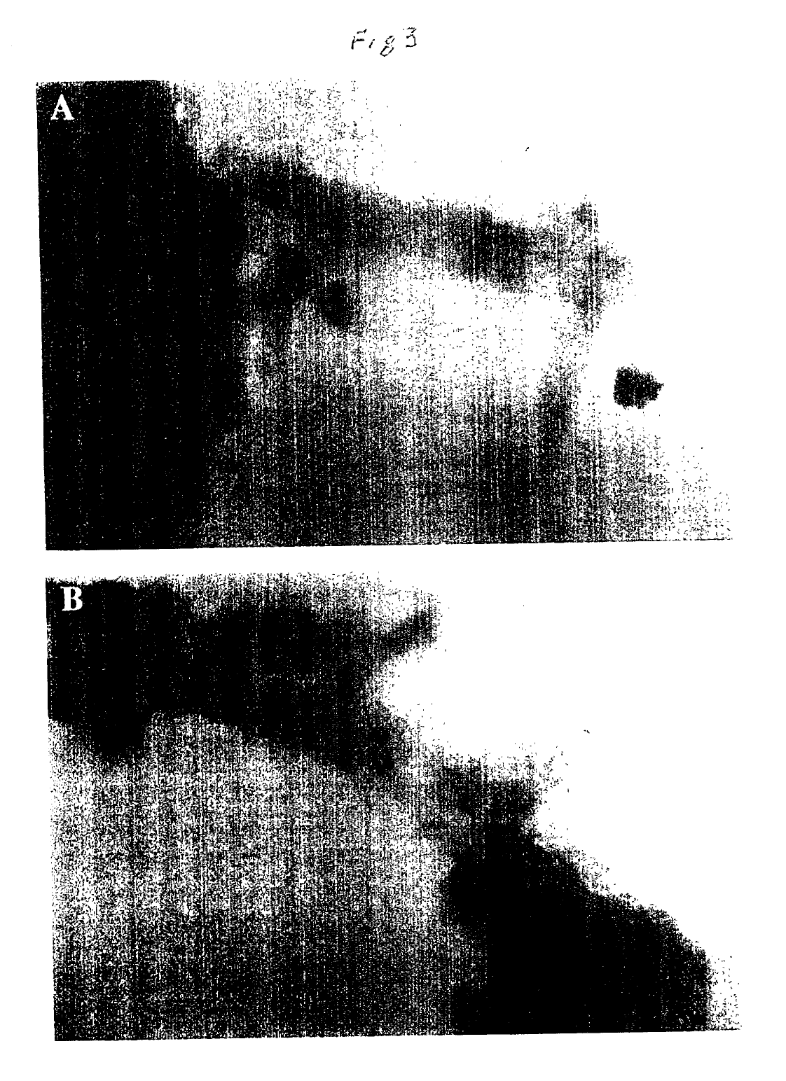

To further confirm the glomeruli-specific pattern of lacZ expression, virus-treated rats (7.5×10¹¹ particles/rat, 15 min infusion into the right kidney) were sacrificed 24 h after viral infusion. The intrarenal arterioles of the right and left kidneys were carefully dissected under direct stereoscopic visualization, fixed in 0.5% glutaraldehyde at room temperature for 20 min, washed with PBS, and stained 1 h for lacZ activity. As shown in FIG. 3, lacZ expression was detected only in glomeruli but not in the intrarenal arterioles in the infused kidney (intrarenal arterioles from right kidney, FIG. 3A; from control left kidbey, FIG. 3B).

### EXAMPLE 3

### Procedure- and Viral-Related Kidney Injury

This example shows that the viral-infusion procedure and subsequent transgene expression result in only mild kidney injury that are well tolerated by the experimental animals.

The virus treated rats (Ad.CBlacZ, 5×10¹¹ particle/ml, 15 min perfusion into the right kidney, flow rate of 0.1 ml/min) were sacrificed at day 3 post-infusion (p.i.) Kidney tissues were fixed in formalin, sectioned and stained for vWF, smooth muscle α-actin, or with Masson Trichrome.

To determine whether the infusion procedure and the virus-mediated transgene expression in glomeruli result in kidney injury, all renal kidney sections from virus-treated rats were carefully examined for histological changes at day 3 and 21 after viral infusion. Histological examination of the H&E and PAS stained sections did not reveal any significant glomerular sclerotic lesions or tubulo- interstitial injury when the infused kidneys (right kidneys) were compared with the contralateral control kidneys (left kidneys) at any time point (Injury scores: 3 days: Infused kidney: 0.99+0.34 vs. control kidney: 0.78+0.12, p>0.05; 21 days: Infused kidney: 1. 23+0.4 vs. control kidney: 1.08+0.2, p>0.05 ). In a similar manner, the immunohistochemical staining with antibodies against vWF and smooth-muscle-a-actin did not show any significant differences in renal glomeruli from virus-infected kidneys at day 3 and day 21, compared to the control kidneys.

Furthermore, Masson and a-actin immunohistochemistry staining did not revealed any evidence of extracellular matrix or collagen accumulation or activation of myofibroblasts respectively in tubulo-interstitium at day 3 and day 21. However, we found a statistically significant increase of PCNA-positive cells in both renal glomeruli and tubules at day 3 (Table 2). The data were obtained in experiments in which virus- treated rats (Ad.CBlacZ, 5×10¹¹ particle/ml, 15 min perfusion into the right kidney, flow rate of 0.1 ml/min) were sacrificed at day 3 and 21 p.i. Kidney tissues were fixed in formalin, sectioned and stained for PCNA. Glomeruli staining was observed at day 3 p.i. and day 21 p.i. in the infused, but not the control kidney.

In addition, we detected an increased recruitment of mononuclear cells, which stained positive with a monocyte-specific antibody (ED1), at day 3 and 21 in both renal glomeruli and tubulo-interstitium of the virus-infused kidneys (Table 2) These data were obtained from experiments in which virus-treated rats (Ad.CBlacZ, 5×10¹¹ particle/ml, 15-min perfusion into the right kidney, flow rate of 0.1 ml/min) were sacrificed at day 3 or 21 p.i. Kidney tissues were fixed in formalin, sectioned and stained with monoclonal antibody ED1.

### EXAMPLE 4

### In Vitro Gene Transfer To Human Renal Cells

This example shows that all human glomerular cell types can be infected by adenovirus in vitro.

To explore the feasibility of transducing human glomerular cells using a similar adenoviral vector, we tested the susceptibility of different human glomerular cell types to adenoviral infection in an in vitro setting. Primary cultures of human glomerular endothelial cells, podocytes, mesangial cells, and renal tubular epithelial were cultured at approximately 80% confluence under identical conditions described in the Methods sections, and infected with Ad.CBlacZ at MOIs ranging from 0.4 to 40 pfu/cell. All cells were fixed 24 h later and stained for lacZ expression. The data shown in FIG. 46 were obtained by experiments in which cultures of the different human kidney cell types were plated in 6-well culture dishes at a density of 5×10⁵ cells/well. Recombinant adenoviruses (Ad.CMVlacZ) were added to the culture medium 24 h later. The cells were fixed 24 h after the infection and stained for β-gal activity. Panel A-D, podocytes infected at an MOI of 0, 0.4, 4 or 40 pfu/cell, respectively. Panel E-H, renal glomerular endothelial cells infected at an MOI of 0, 0.4, 4 or 40 pfu/cell, respectively. Panel I-L, mesangial cells infected at an MOI of 0, 0.4, 4 or 40 pfu/cell, respectively. Panel M-P, renal urinary tubular epithelial cells infected at an MOI of 0, 0.4, 4 or 40 pfu/cell, respectively. Representative pictures are shown. Original magnification 100×.

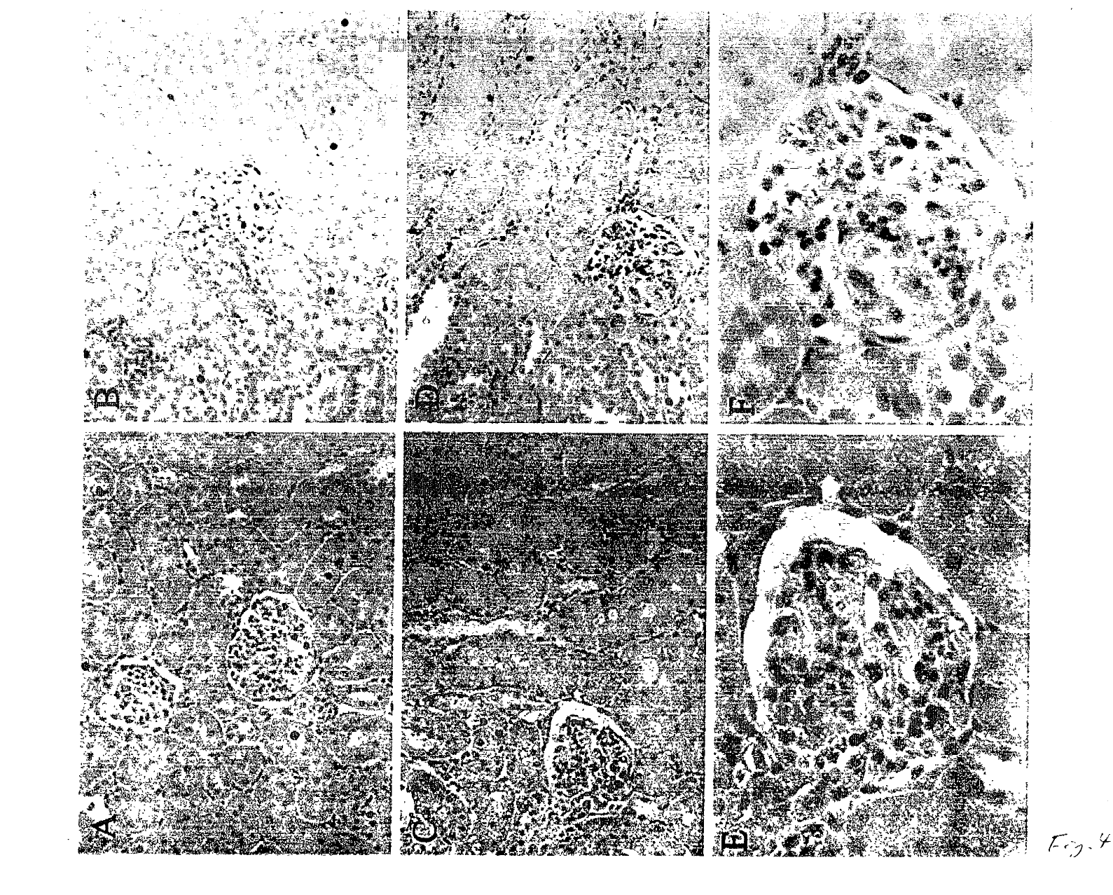

As shown in FIG. 4, all glomerular cell types appeared to be equally susceptible to adenoviral infection with a transduction efficiency of 50% at an MOI of 4 (FIGS. 4A-4L). The renal tubular epithelial cells, however, were less susceptible and required an MOI of 40 to reach 50% transduction (FIGS. 4M-4P).

TABLE I
Adenovirus-mediated gene transfer to rat kidney

| | Viral dose | Perfusion | Sac'd | Lac Z expression | | |
|---|---|---|---|---|---|---|
| Animal No | (particles/rat) | time(min) | at | right kidney | left kidney | liver |
| 1-1 | 1.5 × 10¹¹ | 5 | day 3 post infusion | 0% | 0% | 10% |
| 1-2 | | | | 0% | 0% | 60% |
| 1-3 | | | | 0% | 0% | 30% |
| 2-1 | 1.5 × 10¹¹ | 15 | day 3 post infusion | 0% | 0% | 70% |
| 2-2 | | | | 0% | 0% | 60% |
| 2-3 | | | | 0% | 0% | 40% |
| 3-1 | 7.5 × 10¹¹ | 5 | day 3 post infusion | 0% | 0% | 95% |
| 3-2 | | | | 0% | 0% | 70% |
| 3-3 | | | | 0% | 0% | 80% |
| 4-1 | 7.5 × 10¹¹ | 15 | day 3 post infusion | 30% glomeruli | 0% | 80% |
| 4-2 | | | | 50% glomeruli | 0% | 100% |
| 4-3 | | | | 70% glomeruli | 0% | 100% |
| 5-1 | 7.5 × 10¹¹ | 15 | day 21 post infusion | 10% glomeruli | 0% | 30% |
| 5-2 | | | | 10% glomeruli | 0% | 50% |
| 5-3 | | | | 15% glomeruli | 0% | 40% |

Tissue samples from four animals were examiiied at each time point. Quantification was made by counting lacZ positive cells (in liver) or renal glomeruli in ten microscopic fields with 100× magnification. A lacZ positive glomerulus is defined as a glomerulus that contains at least three lacZ positive cells.

TABLE 2
PCNA and EDI staining of kidney tissues.

| | | Positive cells per microscopic field (100X) | |
|---|---|---|---|
| Staining method/structure | | right kidney (infused) | left kidney (control) |
| PCNA staining: | | | |
| Glomerular | day 3 | 0.77 ± 0.15* | 0.10 ± 0.04 |
| | day 21 | 1.38 ± 0.20* | 0.52 ± 0.12 |
| Tubular | day 3 | 28.20 ± 3.04* | 12.27 ± 0.77 |
| | day 21 | 8.70 ± 0.90 | 6.97 ± 0.64 |
| EDI staining: | | | |
| Glomerular | day 3 | 1.72 ± 0.20* | 1.05 ± 0.15 |
| | day 21 | 2.93 ± 0.26* | 1.18 ± 0.15 |
| Interstitial | day 3 | 17.03 ± 2.56* | 7.07 ± 0.65 |
| | day 21 | 11.50 ± 1.23* | 5.70 ± 0.63 |

*Statistically different from that in control kidney, p < 0.005.

1. A method of infecting the glomerular cells of a kidney of a mammalian subject requiring same with a recombinant adenovirus vector carrying a gene or genes of interest, comprising the step of infusing intra-renal arterially in a single pass through the superior mesenteric artery or renal artery an effective amount of said adenoviral vector into said kidney at an effectively slow rate over an effective period of time, under conditions such that at least 30% of said glomerular cells are infected with said vector.

2. The method according to claim 1, wherein said adenovirus vector carries a control element that preferentially expresses said gene or genes into renal glomerular cells.

3. The method according to claim 1, wherein said kidney is maintained at reduced temperatures during said infusion procedure,

4. The method according to claim 1, further comprising clamping the aorta above and below said superior mesenteric renal artery of said kidney, and infusing through said superior mesenteric renal artery.

5. The method of claim 1, wherein said renal artery is cannulated directly without clamping of said aorta during said infusion.

6. The method of claim 1, wherein said mammal is a rodent, said rate of infusion is about 0.1- 0.5×10¹¹ particles per minute, and said effective period of adenoviral vector infusion is between about 15 and 120 minutes.

7. The method of any one of claims 1 through 6, further comprising concurrent cannulation of the femoral vein through the vena cava into the renal vein so as to direct vector not taken up by renal glomerular cells away from the general circulation.
